(19) United States
(12) Patent Application Publication
(10) Pub. No.: US 2003/0224384 A1
(43) Pub. Date: Dec. 4, 2003

(54) Divide and conquer system and method of DNA sequence assembly

(76) Inventors: Khalid Sayood, Lincoln, NE (US); Hasan H. Otu, Lincoln, NE (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/295,030

(22) Filed: Nov. 13, 2002

Related U.S. Application Data
(60) Provisional application No. 60/350,655, filed on Nov. 13, 2001.

(51) Int. Cl. C12Q 1/68; G06F 19/00; G01N 33/48; G01N 33/50

(52) U.S. Cl. 435/6; 702/20

(57) ABSTRACT

The present invention provides a new algorithm for assembling fragments from a long DNA sequence. The algorithm of the invention solves simultaneously fragment assembly-related problems such as the fragment orientation, overlap and layout phases. This is achieved by clustering fragments with respect to their Average Mutual Information (AMI) profiles using the k-means algorithm.

### FIELD OF THE INVENTION

The present invention relates to a novel algorithm for assembling fragments derived from a long DNA sequence. The algorithm described herein solves simultaneously fragment assembly-related problems such as fragment orientation, overlap and layout phases. The fragment orientation and overlap detection are solved within the clusters, thus reducing the burden of considering a collection of fragments as a whole.

### BACKGROUND OF THE INVENTION

The discovery of restriction enzymes and DNA polymerases around 1970s started the era of DNA sequencing. However, the current technology does not allow sequencing of large contiguous stretches of DNA (greater than a few hundred bases). To try to overcome this problem, the DNA strands are divided into subsequences or fragments utilizing various physical means, such as restriction enzymes, sonication or pressure shearing. A subsequence obtained in, this manner is then sequenced in the standard (5′→3′) direction. This approach is known as the shotgun sequencing method, and is most commonly used in large-scale DNA sequencing projects. Initially, multiple copies of the target DNA are obtained (typical values are between 5-10) followed by sampling of the fragments from such copies. Fragment length is typically between 200 base pairs (bp) and 700 bp, and the number of fragments is in the range of 500 to 2000. The position of the fragments and their respective strand localization are random, however sequencing is always performed in (5′→3′) orientation. Thus, the goal of the shotgun sequencing is to reconstruct the original double-stranded DNA sequence using such sampled fragments. Such reconstruction is possible due to the fact that identifying one strand provides necessary information to identify the other, complementary DNA strand. However, this method of sequencing exhibits limited success with identifying DNA sequences that are 30,000 bp to 100,000 bp in length.

A few supplementary and alternative approaches to shotgun DNA sequencing exist in the art. Among the most common ones are direct sequencing, dual end sequencing and sequencing by hybridization. For other approaches, see, for example, Studier, F. W., Proc. Natl. Acad. Sci. U.S.A., 86:6917-6921, 1989, and Allison et al., Scanning Microsc., 4:517-522, 1990. However, it should be noted that most of these methods are simply alternative approaches to generating fragments, and they still require methods to assemble such fragments. The traditional shotgun approach still has a great deal of appeal because it is economical, parallelizable, and automatable.

Some problems that complicate “fragment assembly” are errors, unknown orientation of the fragments, incomplete coverage of the template, and repeated regions. The simplest forms of errors are known as the base call errors, and they entail base substitutions, insertions, or deletions in the fragments. Base call errors generally occur at about 1%, but can be as high as 5%. The distribution of such errors is not uniform along the sequence; instead, it tends to be greater towards the 3′ ends of the fragments.

To complicate matters further, one generally does not know to which strand of the DNA a given fragment belongs. Since the orientations are not known, one should consider all possible combinations for a collection of n fragments. As the number of such combinations is 2ⁿ, this is not feasible in practice. However, this large number of combinations provides an idea of how difficult the problem of “fragment assembly” is, even when one only considers the complexity introduced by the orientation problem.

The coverage at position i of the target sequence is defined as the number of fragments that include such position. The incomplete coverage occurs when there are positions in the target sequence that are not included in any of the fragments in a given collection. In such case, the target cannot be reconstructed completely, but can be represented as a layout of contiguously covered regions, called contigs. In addition, since the fragments may be aligned arbitrarily, the repeats in the target sequence may cause problems when the length of a repeat exceeds the fragment length, thereby affecting the global solution of the algorithm.

The approaches used previously to achieve the assembly of DNA subsequences in most cases simply attempted to “meld” the fragments together. However, an algorithm for sequence assembly in the most general setting should deal with errors in the fragments, insufficient coverage of the target sequence, unknown orientation and unknown location of fragments. In one prior mathematical model, a formal definition of the sequence reconstruction problem was given for the first time. For notational purposes, let \overline{S} denote the reverse complement of a sequence S, and d(S₁,S₂) be the minimum number of insertions, deletions, and substitutions required to edit sequence S₁ into sequence S₂. d(S₁,S₂) is called the edit distance between S₁ and S₂.

Definition (Sequence Reconstruction Problem): Let T be the target sequence and F′={fᵢ′}₁ⁿ be a collection of fragments sampled from T or \overline{T} at random. Define a new collection of fragments F={fᵢ}₁ⁿ such that fᵢεF is obtained by modifying fᵢ′εF′ with E[d(fᵢ, fᵢ′)]=ε and E[fᵢ]=1. Find a shortest sequence S such that ∀fᵢεF,∃gεS such that

min(d(fᵢ,g),d(\overline{f}i,g))≦ε|g|

where E[ ] denotes expectation and ε is the expected error rate in the fragments. Although the requirement for S to be shortest has no biological motivation, it is a natural condition, considering the principle of parsimony, and it makes the problem mathematically non-trivial. The well known shortest common superstring (SCS) problem in computer science can be reduced to the sequence reconstruction problem with ε′=0. This implies that the sequence reconstruction problem is NP-complete as the shortest common superstring problem is NP-complete. Prior work provides a common approach to fragment assembly by dividing the problem into three phases: overlap, layout, and consensus. See, for example, Peltola et al., Nucleic Acids Research, 7:529-545, 1979. In the first phase, all “acceptable” overlaps between fᵢ and fⱼ and between \overline{f}ᵢ and \overline{f}ⱼ are found. The result of this first phase can be represented as an overlap multi-graph. The second phase consists of computing such an overlap multi-graph and reducing it to an interval graph whose nodes can be interpreted as intervals on a line and there is an edge between two nodes if and only if the corresponding two edges intersect. In the final phase, a consensus sequence is obtained by aligning all fragments that cover the same region.

The problem has also been approached in terms of contigs. Such approach involves processing fragments one at a time and comparing them to the existing layout. A result is one of the three possibilities: 1) a new contig is formed, 2) an old contig is extended, or 3) two contigs are connected. Furthermore, after each iteration the consensus sequence is recomputed. A great majority of available fragment assembly algorithms follow these two basic approaches.

Kececioglu and Myers (1995) studied this approach in four phases, providing a careful formal model as well as exact and approximate algorithms for each phase. The four phases consist of constructing a graph of approximate overlaps between pairs of fragments, assigning an orientation to the fragments, selecting a set of overlaps that induce a consistent layout of the oriented fragments, and merging the selected overlaps into a multiple sequence alignment before voting on a consensus. It was shown that the problems in all but the first phase are NP-complete, and the corresponding approximate solutions were given. As noted in the same publication, the proposed approach artificially separates orientation and layout phases and solves these problems optimally, however without necessarily producing an optimal solution to the combined reconstruction problem. It also suffers a major drawback of applying the shortest common superstring problem to fragment assembly: overcompressing the target. This becomes significant when the target contains repeats longer than the length of an average fragment. As researchers begin to sequence higher organisms, the target sequences become more likely to contain such problematic repeats. This is a common phenomenon seen in the DNA of complex organisms, as discussed in Bell, G. I., Computers Chem., 16:135-143, 1992. The pitfall, which SCS approaches fail to avoid, is that they tend to combine the repeats as the algorithm attempts to find the shortest common superstring, frequently resulting in incorrect formulation in both practice and theory.

Myers (Myers, E. W., J. Comp. Bio., 2:275-290, 1995) defined a new formulation of fragment assembly related to finding a sequence which maximizes the likelihood of the hypothesis that the fragments are sampled over a target with known distribution. The likelihood function is the pdf of the Kolmogorov-Smirnov test statistic for the quality of the similarity between a sample and source distribution. This strategy offers an alternative solution to the layout phase of the traditional SCS approach. Huang introduced the CAP family in which he applied the basic local alignment of Smith and Waterman to compute an overlap. See, for example, Huang, X., Genomics, 10:18-25, 1992, Huang, X., Genomics, 33:21-31, 1996, and Huang et al., Genomics, 9:868-877, 1999. This approach maximized the number of exact matches and errors by trading matches against errors linearly. Both Peltola's and Huang's methods were able to accommodate substitution errors within their objective function. Huang applied the technique of Chang and Lawler for a fast detection of overlapping fragments. This technique enabled him to avoid considering some of the pairs of fragments whose alignment score is below a fixed threshold. The assembler phrap clips the low quality regions (generated by another program phred), using consistent pairwise matches in order to find overlaps and constructs contig layouts. This clipping capability was also included in the final version of the CAP program. The idea of having a fast method for detecting overlaps was also used by Meidanis, however his method utilized the Karp-Rabin string matching algorithm. In both techniques, all fragments were used for comparison, but the improvement lies in using a faster algorithm for finding overlapping fragments. These approaches along with others in the art introduced a new class in the overlap and/or layout phase of the assembly process that is characterized by the use of stochastic search algorithms instead of some other directed methods.

The simulated-tempering Monte-Carlo method has been applied to the sequence assembly problem. It differed from using simulated-annealing for sequence assembly in the fact that it used stochastic moves in temperature. In simulated annealing, an energy function is defined based on the overlaps of the fragments. In addition, this function is minimized by using stochastic reshuffling of the fragments. Thus, the algorithm presented in this method falls in the same category as the above-mentioned approaches. However, it should be noted that the stochastic search process as defined in the Monte-Carlo method did not compare all fragments, which was the case for the above techniques.

Some of the recent approaches were developed by Kim et al. (J. Comp. Bio., pp.163-186, 1999) and Chen et al. (Proceedings of the 8th Symposium on Combinatorial Pattern Matching, pp.206-223, 1997). In Kim et al. publication, fragment overlaps were determined by exact matches of short patterns that were randomly selected from fragment data. The motivation is hybridization fingerprinting, wherein the overlaps between DNA clones are identified using biological short patterns, or “probes”. After the probe matching phase, contigs are formed by comparing the overlap rates of the unmatched fragments with the existing contigs. The significance of an overlap is measured by structured probe matches between the contig and the fragment. The final form of the proposed algorithm is slightly modified to handle the repeats in the target sequence, which are identified by statistical properties obtained from the probe matching phase (e.g. unusually high occurrences of a probe hints a repeat). Following this, the repeat regions are constructed, and the fragments contained in these regions are discarded. The basic algorithm then assembles the remaining fragments. In the other approach suggested by Chen et al., the application of suffix trees and suffix arrays in overlap detection is investigated.

As can be seen, DNA fragment assembly still presents a significant challenge in terms of available algorithms capable of performing such fragment assembly, particularly in cases of long DNA sequences. Thus, novel and/or improved algorithms for DNA sequence assembly are needed.

### SUMMARY OF THE INVENTION

Among the aspects of the present invention is a provision of a method for assembling subsequences of a DNA sequence. Briefly, the method comprises:

assigning a numerical characterization to each subsequence, wherein each subsequence comprises a set of numbers;

clustering the sets; and

aligning the sets to form a consensus sequence for each cluster.

The method may further comprise defining a vector for each subsequence, wherein each vector possesses coordinates corresponding to the numerical characterizations of one subsequence. In this embodiment, the step of clustering comprises clustering the vectors and step of aligning comprises comparing the vectors in each cluster and aligning the subsequences of corresponding compared vectors to form a consensus sequence for each cluster.

In another embodiment, a system for assembling subsequences of a DNA sequence is provided. Said system comprises:

a database of the subsequences to be assembled; and

a processor capable of accessing the subsequences in the database and having software for:

assigning a numerical characterization to each subsequence, each numerical characterization comprising a set of numbers,

clustering the sets, and

aligning the sets to form a consensus sequence for each cluster.

In still another embodiment, a system for assembling subsequences of a DNA sequence comprises:

a database of the subsequences to be assembled; and

a processor capable of accessing the subsequences in the database and having software for:

assigning a numerical characterization to each subsequence, each numerical characterization comprising a set of numbers,

defining a vector for each subsequence, each vector having coordinates corresponding to the numerical characterization of one subsequence,

clustering the vectors,

aligning the vectors, wherein aligning comprises comparing the vectors in each cluster and aligning the subsequences of corresponding compared vectors to form a consensus sequence for each cluster.

Other objects and features will be in part apparent and in part pointed out hereinafter.

### BRIEF DESCRIPTION OF THE DRAWINGS

FIG. 1 illustrates the spanning tree used in multiple alignment wherein the branches of the maximum spanning tree are in bold.

FIG. 2A (left side of FIG. 2) illustrates a cluster with four subsequences above the horizontal line and the corresponding consensus sequence below the horizontal line.

FIG. 2B (right side of FIG. 2) illustrates a cluster with three subsequences above the horizontal line and the corresponding consensus sequence below the horizontal line.

FIG. 3 illustrates a cluster with the two consensus sequences of FIGS. 2A and 2B above the horizontal line and the corresponding consensus sequence below the horizontal line.

FIG. 4 illustrates the actual alignment, corresponding interlacation and the alignment implied by the interaction of two sequences labeled (*) and (∘).

FIG. 5 illustrates three sequences labeled (*), (∘) and (⋄) in which the pairwise interlacations are combined and in which the multiple alignment implied by the combined interlacations is generated.

### ABBREVIATIONS AND DEFINITIONS

To facilitate understanding of the invention, a number of terms are defined below:

“AMI profile” as used herein refers to an average mutual information profile.

As used herein, “bp” refers to a “base pair”.

As used herein, A, C, T, and G refer to nucleotides adenine, cytosine, thymine and guanine, respectively.

### DETAILED DESCRIPTION OF PREFERRED EMBODIMENTS

The present invention relates to a method and system which solves the orientation, overlap, and layout phases of DNA sequence assembly simultaneously. Most of the existing algorithms put a significant amount of computational burden in the overlap phase, wherein each fragment is compared with all the remaining fragments and their reverse complements. It has been found that this appears to be unnecessary, at least in some analyses, as the number of “similar” fragments is in the order of coverage which is much smaller than the number of fragments. The method of the present invention avoids this drawback by clustering the fragments before exploring overlaps. Specifically, an average mutual information (AMI) profile is used to measure the degree of “closeness” between fragments, and a k-means algorithm is employed to generate the clusters. As a result, the method described herein is a powerful technique, when taking into account that fragments coming from the same regions of the target sequence have similar AMI profiles. Moreover, AMI profiles are robust to errors and remain unchanged when calculated for the reverse complements of fragments. Therefore, the orientation and overlap problems are solved within the clusters, which already contain fragments coming from the same region of the target.

In addition, a problem frequently encountered during sequencing is the presence of repeats. When there are repeating regions in the target sequence, the fragment assembly algorithms tend to overcompress the final consensus by combining the repeat regions. Furthermore, the assembly programs are helpless when the length of the repeat region is greater than the fragment length. In contrast to this, the algorithm described herein handles the problem by repeatedly running the program and discarding the cases where the final consensus is short of the expected target length.

Described below is one preferred embodiment of a method according to the invention.

### AVERAGE MUTUAL INFORMATION (AMI)

For a sequence S, the average mutual information function is defined as:

$$I ( r ) = {\sum }_{i , j} {p}_{ij} ( r ) {log}_{2} ( \frac{{p}_{ij} ( r )}{{p}_{i} {p}_{j}} )$$

where pᵢ is the density for symbol i and pᵢⱼ(r) is the joint probability of observing symbol i and j separated by a distance r. I(r) is the amount of information symbol i carries about symbol j at a distance r. For a random sequence with infinite length I(r) is 0 for all r. AMI profiles of DNA sequences have been studied in the field of bioinformatics for various purposes. For example, the AMI profiles have been used to recognize the coding regions in DNA. Luo et al. use I(r) to study statistical correlation of nucleotides in DNA. Recently, AMI profiles of genomic sequences have been used for analysis of evolutionary history.

In the present invention, the vector Aᵢ is calculated for each fragment f₁, where Aᵢ(r)=I(r) and r=1, . . . , R by obtaining the required densities from fᵢ. Following such calculation, R-dimensional vectors are obtained, namely the AMI profiles associated with each fragment. These vectors are clustered using the k-means algorithm.

### CLUSTERING

Clustering can be defined as grouping the elements of a set subject to a certain measure of similarity. In particular, k-clustering partitions the given set into k non-empty sets. The k-means algorithm represents a special case of the more general k-clustering algorithm. This algorithm is utilized along the lines of Vector Quantization (see, e.g., K. Sayood, “Introduction to Data Compression”, Second Edition, Morgan Kauffman Academic Press, San Francisco, 2000), which is a frequently used method in Data Compression. Given N vectors

$${\{ {A}_{i} \}}_{i}^{N} = 1 ,$$

a threshold λ and a perturbation vector e(B), J clusters are obtained as follows:

1. set

$${B}_{1} = \frac{1}{N} {\sum }_{i = 1}^{N} {A}_{i} ,$$

2. For each Bₖ, k=1, . . . ,N, if k=J, stop; otherwise, let

Bₖ₊₁=e(Bₖ),i=1, . . . ,k. D₀=0,

3. determine Cⱼ={Aₙ:d(Aₙ,Bⱼ)<d(Aₙ,Bᵢ)∀i≠j},j=1, . . . ,2k.

4. determine

$${D}_{n} = {\sum }_{j = 1}^{2 k} {\sum }_{i = 1}^{\vert {c}_{j} \vert} d ( {B}_{j} , {A}_{ij} ) ,$$

where Aᵢⱼ is the i^{th} vector in the j^{th} cluster, Cⱼ;

5. if

$$\frac{{D}_{n} - {D}_{0}}{{D}_{n}} < λ ,$$

go to step 2, otherwise continue;

6. for D₀=Dₙ, find new representation vectors Bⱼ that are the average value of the vectors in the j^{th} cluster Cⱼ; go to step 3;

where d(A,B) is the Euclidean distance between the vectors A and B. The algorithm is given for the case where the target number of the clusters, J, is a power of 2. The modification for other cases can simply be done by not perturbating all of the representing vector Bⱼ at step 2.

### PROCESSING THE CLUSTERS

The clustering algorithm used in the previous section partitions the fragments into J clusters using the AMI profiles of the fragments as a measure of similarity. Therefore, the fragments in the same cluster are likely to come from the same region of the target sequence. However three issues that still need to be addressed include clustering errors, orientation and layout. A clustering error occurs if, in a given cluster, there exists more than one group of fragments whose elements do not truly overlap. More precisely, if there are at least two groups of fragments C_{J}¹ and Cⱼ² in a given cluster Cⱼ, such that

1. for all fεCⱼⁱ, there exists a gεCⱼⁱ, such that f and g truly overlap,

2. for all fεCⱼⁱ, there exists no Cⱼ^{k}, such that a fragment in C_{J}^{k} truly overlaps with f,

there has been a clustering error. These subgroups, Cⱼⁱ, in the cluster Cⱼ need to be identified. This is done by calculating the scores of the pairwise alignments of the fragments in a cluster. Since the orientation is unknown, it is important to align both fₘ and \overline{f}ₘ with fₙ, where \overline{f}ₘ denotes the reverse complement of fₘ and m=1, . . . , |C|−1, n=m+1, . . . , |C_{J}|. Thus, there are |C_{J}||Cⱼ−1| comparisons. Since

$$E [ \vert {C}_{j} \vert ] = \frac{N}{J} ,$$

this calculation is feasible. More specifically, let the fragments in a given cluster be

$${\{ {f}_{ i} \}}_{i = 1}^{\vert {C}_{j} \vert} .$$

The subgroups are generated as follows:

1. put fₖ in Cⱼ^{k}, k=1, . . . ,|Cⱼ|, set i=1,

2. if s(fᵢ,fⱼ)>λ(fᵢ,fⱼ), or s(fᵢ\overline{,}fⱼ)>λ(fᵢ\overline{,}fⱼ) combine the subgroup that contains fⱼ with the subgroup that contains fᵢ, do this for j=i+1, . . . , |Cⱼ|,

3. increment i; if i=|Cⱼ|, stop; otherwise, go to step 2,

where s(fᵢ,fⱼ) is the score of the optimal semiglobal alignment (i.e. we ignore the gaps in the extremities of either sequence) between the fragments fᵢ and fⱼ. λ(fᵢ,fⱼ) is a threshold function which indicates the minimum score of the optimal semiglobal alignment between fᵢ and fⱼ such that the score has at least (1−ε) % significance, where ε is the error rate in sequencing imposed by the shotgun phase. Calculation of λ( ) is addressed further below. An example cluster, C₁, is shown in Table 1. Table 2 shows the scores of the pairwise optimal semiglobal alignment. After examining Table 2, the algorithm puts fragments 1, 2, 3, 7 and 8 in the new cluster, C₁¹, and the fragments 4, 5, 6 and 9 in the new cluster, C₁². These two clusters are shown in Table 3.

TABLE 1
A Sample Cluster
Cluster C₁

| # | orientation | position |
|---|---|---|
| 1 | F | 5587-6062 |
| 2 | F | 5545-5964 |
| 3 | R | 5251-5737 |
| 4 | R | 1608-2085 |
| 5 | F | 1257-1796 |
| 6 | R | 1262-1798 |
| 7 | R | 5489-6057 |
| 8 | F | 4900-5425 |
| 9 | F | 1303-1811 |

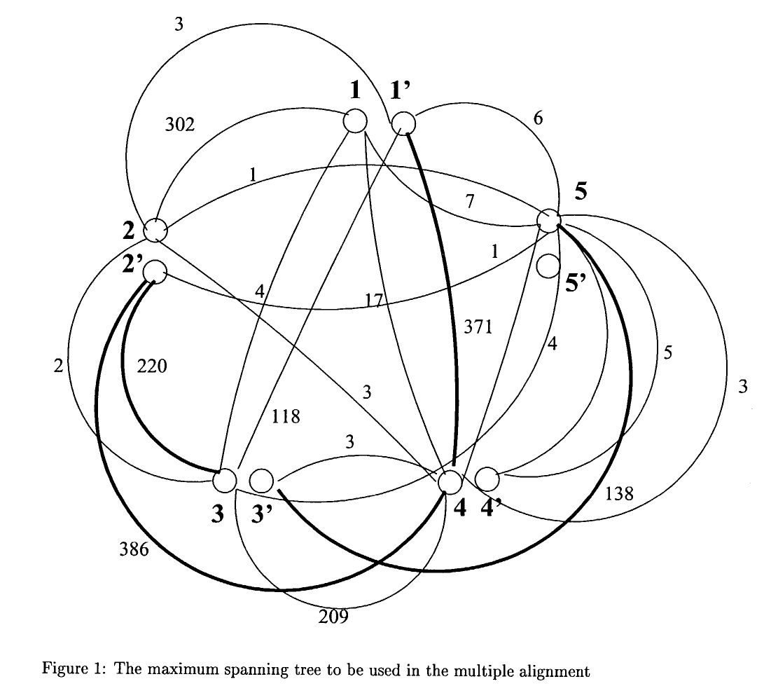

The next requirement is to find a consensus sequence for each subgroup born from the cluster Cⱼ. It should be noted that the scores for the optimal semiglobal alignment of each pair in the subgroup are already known. Accordingly, this induces a graph with 2T nodes and 2T(T−1) edges, where T is the number of fragments in the subgroup and the weight of an edge is the score of the alignment. A maximum spanning tree is needed for the induced graph, where the tree contains T nodes. The multiple alignment strategy taken to find a consensus sequence is explained below. This can be done by applying Prim's algorithm in O(T+T log T) time (for Prim's algorithm, see, e.g., R. C. Prim, Bell System Tech. J., Vol. 36, pp. 1389-1401, 1957). It should be noted that although the induced graph contains 2T nodes (as both f and \overline{f} are represented as nodes), a maximum spanning tree of T nodes is needed where each node is either fᵢ or \overline{f}ᵢ. In order to obtain the multiple alignment, the only needed information is the relative orientation of fragments as a branch is added to the maximum spanning tree. The reason for this is that the optimal alignment between fᵢ and \overline{f}ⱼ implies the optimal alignment between \overline{f}ᵢ and fⱼ with the same score. The overlap graph for cluster C₁¹ is illustrated in FIG. 1, where the branches of the maximum spanning tree are represented in bold. In the same figure, the node 1 represents the fragment #1 in the cluster C₁¹, the node 1′ represents the reverse complement of the fragment #1, and so on. Note that the maximum spanning tree consists of the edges obtained by connecting the nodes 1′⇄4,4⇄2′,2′⇄3 and 3′⇄5′. However, as the algorithm picks the orientation of the first sequence as the reference, the actual pairwise alignments that are used in the multiple alignment phase are 1⇄4′,4′⇄2,2⇄3′ and 3⇄5. Also note that both the sequence and relative orientation of these pairs are arbitrary, and do not affect the consensus sequence.

TABLE 2

| # | 2 | 3 | 4 | 5 | 6 | 7 | 8 | 9 |
|---|---|---|---|---|---|---|---|---|
| 1 | 302 | 4 | 2 | 1 | 5 | 17 | 9 | 3 |
| 2 | | 2 | 2 | 4 | 3 | 3 | 1 | 5 |
| 3 | | | 4 | 2 | 2 | 209 | 4 | 2 |
| 4 | | | | 3 | 156 | 3 | 3 | 3 |
| 5 | | | | | 9 | 3 | 10 | 401 |
| 6 | | | | | | 5 | 4 | 6 |
| 7 | | | | | | | 3 | 2 |
| 8 | | | | | | | | 2 |
| 1 | 3 | 118 | 2 | 2 | 2 | 371 | 6 | 1 |
| 2 | | 220 | 1 | 2 | 4 | 386 | 1 | 3 |
| 3 | | | 3 | 2 | 4 | 3 | 138 | 2 |
| 4 | | | | 165 | 4 | 2 | 2 | 166 |
| 5 | | | | | 443 | 5 | 1 | 7 |
| 6 | | | | | | 2 | 4 | 400 |
| 7 | | | | | | | 5 | 2 |
| 8 | | | | | | | | 6 |

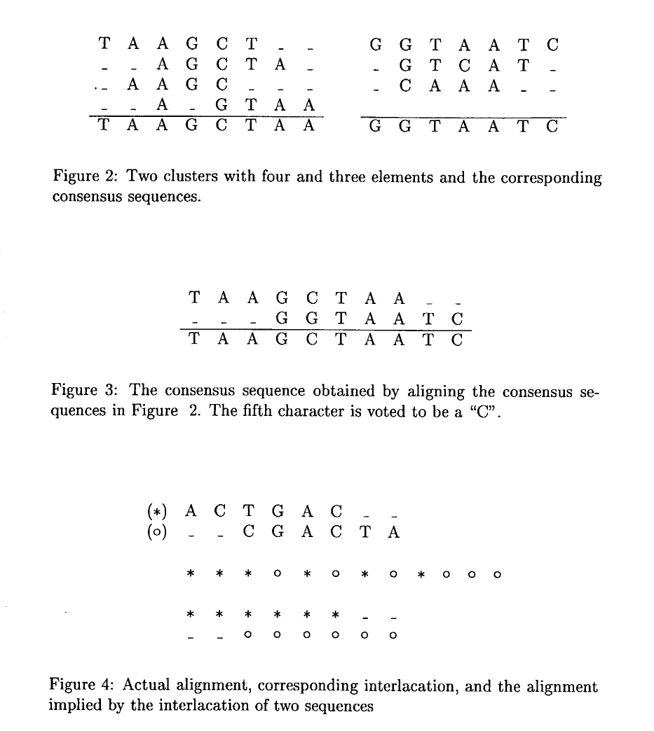

Once a multiple alignment is obtained, the characters of the consensus sequence are determined by a character that receives the maximum vote at the corresponding column of the multiple alignment. The gaps that result from the extremities of any sequence are not considered in the voting. Although the characters of the consensus sequence are fixed, the vote of each character at a given position is kept. Such information is used in the voting process of the multiple alignment of the consensus sequences. The motivation for this can be illustrated as follows. By way of example, consider the consensus sequences in FIG. 2 that are aligned with each other in the next iteration.

TABLE 3
The two new clusters C₁¹ and C₁² born from the
parent cluster C₁

| | | |
|---|---|---|
| # | orientation | position |

Cluster C₁¹

| | | |
|---|---|---|
| 1 | F | 5587-6062 |
| 2 | F | 5545-5964 |
| 3 | R | 5251-5737 |
| 7 | R | 5489-6057 |
| 8 | F | 4900-5425 |

Cluster C₁²

| | | |
|---|---|---|
| 1 | R | 1608-2085 |
| 2 | F | 1257-1796 |
| 3 | R | 1262-1798 |
| 4 | F | 1303-1811 |

The fifth character of Consensus Sequence 1 is fixed as a “C” and the second character of Consensus Sequence 2 is fixed as a “G”. However, since the algorithm keeps the number of characters that had spanned these columns, when these two columns of the consensus sequences are aligned, it is apparent that “C” in the first sequence is in fact three “C”s and a “G”, and similarly, the “G” in the second sequence is in fact two “G”s and a “C”. This is shown in FIG. 3. Hence, the consensus sequence in the second iteration fixes that column as a “C” as it should be. This property enables the algorithm to perform a multiple alignment on a very fine scale, which has generally not been the case with the existing algorithms. As the algorithm described herein processes the fragments in clusters, it is able to do so quickly since the number of fragments in a cluster is relatively small. However, the consensus sequence can still be fine-tuned as the next iteration is applied in the algorithm by clustering the consensus sequences of the previous step.

### RECURSION

The consensus sequences of the second generation clusters can be considered as a new collection of fragments. Thus, the same procedure that was applied to the original collection of fragments is repeated. For example, assuming that we have J₁ new clusters, and letting the consensus sequence of the i^{th} cluster be fᵢ, 1≦i≦J₁, the AMI profile can be computed for each fᵢ and the k-means algorithm can be repeated. In the next step, each cluster is processed as explained above.

This recursion process is repeated until there is one cluster left or until no new cluster is born after all clusters are processed. In the first case, a final consensus sequence for the target is obtained, whereas in the second case, a number of contigs which can be ordered arbitrarily is obtained.

### SIGNIFICANCE OF SEMIGLOBAL ALIGNMENT SCORES

An alignment of two sequences is obtained by inserting gaps in such sequences so that the sizes of the sequences become identical and no two gaps occur at the same place. Any alignment can be scored with a given scoring scheme. More precisely, if the sequences are defined over an alphabet A (assume A as an extended alphabet that contains the gap symbol “_”), the function Φ: A×A−{(_,_)}→R is a scoring scheme. The score of the alignment <S′|T′> of the sequences S and T is ΣᵢΦ(S′[i],T′[i]), where the upper limit of the summation is at most |S|+|T| and S′ and T′ are the sequences S and T padded with gaps respectively. A scoring scheme is called a linear scoring scheme if the function Φ( ) is symmetrical with respect to its arguments and Φ(a,_)<0∀aεA−{_}. In semiglobal alignments, the alignments are scored such that some of the end or beginning gaps are ignored. The type of semiglobal alignment that is useful for the purposes of the present invention is one in which there is no penalty for the gaps in the extremities of either sequence. To evaluate the significance of such a semiglobal alignment, the following problem needs to be solved:

Problem: Given a linear scoring scheme Φ( ),

$$ψ ( a , b ) = \{ \begin{matrix}e , & if & a = b \\ f , & if & a \neq b , \\ g , & if & a = \_ or b = \_\end{matrix}$$

two sequences S and T with |S|=m, |T|=n, and δ, find B such that P(A≧B)≦δ, where A is the score of the optimum semiglobal alignment between S and T.

Solution: Note that in practice δ<<1 is preferably used since it is desirable that the overlap between the fragments be highly significant. This implies a relatively large B, thus few mismatches and gaps in the overlapping region are assumed. Therefore, the problem is solved for a case with no gaps. Without loss of generality, it can be assumed that the scoring scheme is normalized so that the score of a match is 1. Furthermore, it can be assumed that the elements of the sequences are independent and identically distributed (i.i.d.) with uniform distribution and belong to the alphabet {A,T,C,G}. Let p=min(m,n). Consider the upper left half of a p×p matrix, P, including the auxiliary diagonal. In other words, consider the cells pᵢⱼ such that 0≦i≦p and 0≦j≦p−i. Now in this upper half, consider the set of cells Pₖ={pᵢⱼ: i+j=k}, 0≦k≦p. For a fixed k, Pₖ consists of the cells that lie parallel to the auxiliary diagonal. Let pᵢⱼεPₖ represent an overlap of length k with i mismatches and j matches. Given an overlap k, the probability of the cell pᵢⱼεPₖ is

$$P ( {p}_{ij} ) = ( \begin{matrix}k \\ i\end{matrix} ) {( \frac{3}{4} )}^{i} {( \frac{1}{4} )}^{j}$$

with the score j+fi associated with it. Note that for a fixed k,Σₚ_{ᵢⱼ}_{εP}_{ₖ}P(pᵢⱼ)=1. Now, for a given B′ we can calculate P(A<B′) as follows:

$$P ( A < {B}^{′} ) = {\prod }_{k = 0}^{P} P ( s ( {P}_{k} ) < {B}^{′} )$$

where s(Pₖ) is the distribution defined as the score of the overlap given k. Hence

$$P ( s ( {P}_{k} ) < {B}^{′} ) = {\sum }_{i = {I}_{k}}^{k} ( \begin{matrix}k \\ i\end{matrix} ) {( \frac{3}{4} )}^{i} {( \frac{1}{4} )}^{( k - i )}$$

where

$${I}_{k} = ⌊ \frac{{B}^{′} - k}{f - 1} ⌋ + 1.$$

Chernoff Bound can be applied to estimate P(s(Pₖ)<B′). Let Y be the sum of k i.i.d. random variables

$${\{ {X}_{i} \}}_{i = 1}^{k}$$

with

$$P ( {X}_{i} = 1 ) = \frac{1}{4} and P ( {X}_{i} = 0 ) = \frac{3}{4} .$$

Note that P(s(Pₖ)<B′)=P(Y≧Iₖ). Applying the Chernoff Bound, we have

$$P ( Y \geq {I}_{k} ) \leq {min}_{s > 0} {ⅇ}^{- {sI}_{k}} E [ {ⅇ}^{sY} ]$$

On the other hand,

$$E [ {ⅇ}^{sY} ] = E [ {ⅇ}^{s {\sum }_{i = 1}^{k} {X}_{i}} ] = E [ {\prod }_{i = 1}^{k} {ⅇ}^{{sX}_{i}} ] = {\prod }_{i = 1}^{k} E [ {ⅇ}^{{sX}_{i}} ]$$

Now, since

$$E [ {ⅇ}^{{sX}_{i}} ] = \frac{3}{4} + \frac{1}{4} {ⅇ}^{s} , ∀ i ,$$

$$P ( Y \geq {I}_{k} ) \leq {min}_{s > 0} {{ⅇ}^{- {sI}_{k}} ( \frac{3}{4} + \frac{1}{4} {ⅇ}^{s} )}^{k} .$$

The minimum of

$${{ⅇ}^{- {sI}_{k}} ( \frac{3}{4} + \frac{1}{4} {ⅇ}^{s} )}^{k}$$

is achieved when

$${ⅇ}^{s} = \frac{3 {I}_{k}}{k - {I}_{k}} .$$

Therefore,

$$P ( s ( {P}_{k} ) < {B}^{′} ) = P ( Y \geq {I}_{k} ) \leq {( \frac{3 {I}_{k}}{k - {I}_{k}} )}^{- {I}_{k}} {( \frac{3}{4} + \frac{3 {I}_{k}}{4 ( k - {I}_{k} )} )}^{k} .$$

It is desirable to find the expression for

$${\prod }_{k = 0}^{p} P ( s ( {P}_{k} ) < {B}^{′} ) ,$$

since P(A≧B′)≦δ implies P(A<B′)≧1−δ and,

$$P ( A < {B}^{′} ) = {\prod }_{k = 0}^{p} P ( s ( {P}_{k} ) < {B}^{′} ) \leq {\prod }_{k = 0}^{p} {( \frac{3 {I}_{k}}{k - {I}_{k}} )}^{- {I}_{k}} {( \frac{3}{4} + \frac{3 {I}_{k}}{4 ( k - {I}_{k} )} )}^{k}$$

It is roughly assumed that the bound is actually equal, we need to find B′ for which

$${\prod }_{k = 0}^{p} {( \frac{3 {I}_{k}}{k - {I}_{k}} )}^{- {I}_{k}} {( \frac{3}{4} + \frac{3 {I}_{k}}{4 ( k - {I}_{k} )} )}^{k} = 1 - δ$$

It is known that Iₖ is of the form ak+b. Substitute this,

$$log ( 1 - δ ) = {\sum }_{k = 0}^{p} ( - ak - b ) log ( \frac{3 ( ak + b )}{k - ak - b} ) + k log ( \frac{3}{4} + \frac{3 ( ak + b )}{4 ( k - ak - b )} )$$

Noting that

$$k log ( \frac{3}{4} + \frac{3 ( ak + b )}{4 ( k - ak - b )} ) = k log [ \frac{3}{4} ( 1 + \frac{ak + b}{k - ak - b} ) ] = k log ( \frac{3 k}{4 [ ( 1 - a ) k + b ]} )$$

we have

$$log ( 1 - δ ) = {\sum }_{k = 0}^{p} ( - ak - b ) log 3 - ( ak + b ) log ( ak + b ) + ( ak + b ) log [ ( 1 - a ) k - b ] + k log ( \frac{3}{4} ) + k log k - k log [ ( 1 - a ) k - b ]$$

hence

$$c = {\sum }_{k = 0}^{p} k log k - ( ak + b ) log ( ak + b ) - [ ( 1 - a ) k - b ] log [ ( 1 - a ) k - b ]$$

where

$$c = log ( 1 - δ ) + \frac{p ( p + 1 )}{2} log 4 - \frac{p [ ( p + 1 ) ( 1 - a ) - 2 b ]}{2} log 3$$

and roughly,

$$a = \frac{- 1}{f - 1} , b = \frac{{B}^{′}}{f - 1} + 1.$$

### MULTIPLE ALIGNMENT

The multiple alignment strategy described herein is based on the observation that any alignment between two sequences can be represented by an interlacation of the two sequences. This is illustrated in FIG. 4. This observation can be generalized to the case of multiple alignment. In other words, any multiple alignment of n sequences can be represented as an interlacing of these sequences. This interlacing can be obtained from pairwise interlacings. Two pairwise interlacings can be combined into one interlacing as long as the two interlacings that were started with have a sequence in common. For the purposes of the present invention, described herein is a technique to combine the initial pairwise interlacings into one interlacing of all of the sequences. Moreover, as illustrated herein, this can be achieved using a maximum spanning tree over the graph where nodes represent sequences and weight of the edges represent score of the alignment between the two sequences it connects.

In general, the multiple alignment technique comprises aligning the consensus sequences in a cluster in which there is an interlacing of the sequences in each cluster according to the following algorithm:

1. Find the pairwise alignments of the sequences in the cluster.

2. Represent each alignment as an interlacing of the two sequences involved (FIG. 4).

3. Create a graph (FIG. 1) where nodes represent sequences and the weight of an edge connecting two nodes represents the score of the alignment between the sequences (see Table 2).

4. Find a maximum spanning tree of this graph.

5. Combine the pairwise alignment implied by each edge with the pairwise alignment implied by the following edge while moving through the graph.

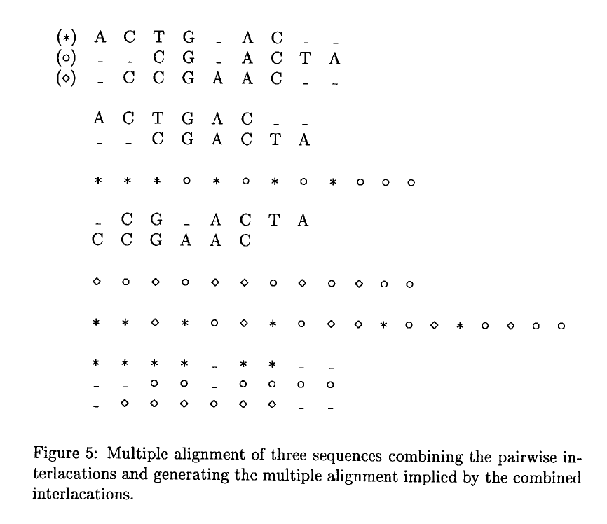

A multiple alignment is a natural generalization of this approach. However, in order to combine two interlacations one common sequence in both interlacations is needed, as shown in FIG. 5. Note that there is a one-to-one correspondence between any such combined interlacation and a multiple alignment. Therefore, if we want to construct a multiple alignment of n sequences, we need a tree, wherein the nodes represent the sequences, a branch between node i and node j implies that the pairwise alignment between sequence i and sequence j is to be used in constructing the multiple alignment, and the weight of the branch implies the score of that alignment. In order to increase the quality of the multiple alignment, the sum of the weights of the branches needs to be increased in such tree. Hence, the optimum solution is obtained by using a maximum spanning tree. Given n sequences in a cluster, the algorithm first generates the tree where the weights of the edges are the scores for the optimum semiglobal alignment between the corresponding sequences. Subsequently, it calculates the maximum spanning tree and finds the multiple alignment using the pairwise alignments provided by such tree.

This invention provides a novel method for solving the fragment assembly problem. Instead of clustering all fragments as a whole, the method provided herein uses the AMI profiles of fragments as a measure of similarity. Such measure is efficient due to the fact that AMI profiles are robust to errors and remain unchanged when calculated for the reverse complement of a sequence. By using a divide-and-conquer algorithm of the present invention, it is possible to process feasible numbers of fragments at a time and calculate the consensus sequence on a finer scale in a shorter time. The simulation results, as presented in the Example, appear promising both for artificial and real data sets. As illustrated herein, the algorithm reconstructs the target sequence with over 99% similarity and within 2% of its length using a coverage of five and an error rate of 5%.

### EXAMPLE

The following example is intended to provide illustrations of the application of the present invention. The following example is not intended to completely define or otherwise limit the scope of the invention.

The algorithm of the invention was tested both on artificial and real data. The target sequences used as artificial data sets were sequences of 50,000 bp and the elements were randomly chosen from the set {A,C,T,G}. Random fragments were then sampled such that the average length of the fragments was 500 bp, and the starting position of the fragments was uniformly distributed along the target. The sampling process was carried out until the total length of the fragments exceeded five times the length of the target. This resulted in a coverage of five. About half of the fragments were replaced by their reverse complements and the fragments were modified such that k+l+m≈ε(n+k−l), where n is the length of the fragment subject to k insertions, l deletions and m substitutions. ε is the introduced error rate.

The target sequences used as real data sets were the first 50,000 bases of yeastl and yeast2 (Gen Bank Accession numbers X59720 and D50617, http://www.ncbi.nlm.nih.gov). These sequences were processed in the same manner as the artificial sequences. The length of the AMI profile vector used in simulations was 16. In each case, the clustering algorithm started with 64 clusters and the number of clusters was halved at each iteration. There were 6 total iterations for each case. The threshold used in the k-means algorithm was 0.001, and the perturbation vector was a constant vector of 0.05, i.e. e(Bₖ)=Bₖ+ν, where ν is the constant vector. Experimenting with these parameters had little or no effect on the final answer.

The results are tabulated in Table 4, where |C| denotes the length of the final consensus sequence and m denotes the number of matches between the target sequence and the final consensus sequence.

The results show that in instances when no error is introduced, the algorithm is able to construct the target sequence with 100% similarity in all cases. In cases when 5% error is introduced, the similarity between the target sequence and the final consensus sequence is over 99% and the length of the final consensus sequence is within 2% of the target sequence. In these results, this was found to apply for all cases. It should be noted that in practical applications the coverage is usually more than five and the error rate is less than 5%.

TABLE 4
Simulation results

| | | | ∈ = 0 | | ∈ = 0 | |
|---|---|---|---|---|---|---|
| Sequence | length | #fragments | \|C\| | m | \|C\| | m |
| random1 | 50,000 | 502 | 50,000 | 50,000 | 50,386 | 49,817 |
| random2 | 50,000 | 499 | 50,000 | 50,000 | 50,435 | 49,756 |
| yeast1 | 50,000 | 498 | 50,000 | 50,000 | 50,712 | 49,574 |
| yeast2 | 50,000 | 503 | 50,000 | 50,000 | 50,842 | 49,650 |

The simulation results indicate that the algorithm of the present invention is a powerful tool to divide the fragment assembly problem and solve the phases discussed in traditional approaches within the groups. As illustrated in Table 1, the AMI profiles successfully distinguish fragments coming from different regions. By processing the clusters, the algorithm described herein can fix the containment, which is a consequence of the fragments coming from distinct regions of the target, and place these fragments in new clusters as shown in Table 3. Furthermore, this approach solves the orientation problem due to the fact that the fragments in a processed cluster have consistent orientation. Thus, calculating the multiple alignment within a processed cluster enables one to perform the final phase of the traditional approaches on a much finer scale. In contrast to our algorithm, such calculation appears as an additional step in the existing algorithms where they try to refine the consensus sequence.

In light of the detailed description of the invention and the examples presented above, it can be appreciated that the several aspects of the invention are achieved.

The above detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method of assembling subsequences of a DNA sequence comprising the steps of:
assigning a numerical characterization to each subsequence, each numerical characterization comprising a set of numbers;
clustering the sets; and
aligning the sets to form a consensus sequence for each cluster.

2. The method of claim 1 further comprising the step of:
defining a vector for each subsequence, each vector having coordinates corresponding the numerical characterizations of one subsequence;
wherein the step of clustering comprises clustering the vectors; and
wherein the step of aligning comprises comparing the vectors in each cluster and aligning the subsequences of corresponding compared vectors to form a consensus sequence for each cluster.

3. The method of claim 1 further comprising aligning the consensus sequences to reconstruct the DNA sequence.

4. The method of claim 3 wherein the step of aligning the consensus sequences comprises aligning the sequences in a cluster through a multiple alignment technique in which there is an interlacing of the sequences in each cluster.

5. The method of claim 4 wherein the multiple alignment technique is performed according to the following algorithm comprising the steps of:
finding the pairwise alignments of the sequences in the cluster;
representing each alignment as an interlacing of the two sequences involved;
creating a graph where nodes represent sequences and the weight of an edge connecting two nodes represents the score of the alignment between the sequences;
finding a maximum spanning tree of this graph; and
combining the pairwise alignment implied by each edge with the pairwise alignment implied by the following edge while moving through the graph.

6. The method of claim 1 further comprising clustering consensus sequences to reconstruct the DNA sequence.

7. The method of claim 1 further comprising obtaining the subsequences by a shotgun sequencing method selected from the group consisting of digestion with restriction enzyme(s), sonication and pressure shearing.

8. The method of claim 1 further comprising obtaining the subsequences by a direct sequencing selected from the group consisting of dual end sequencing and sequencing by hybridization.

9. The method of claim 1 wherein the step of clustering comprises determining a profile for each subsequence and clustering the subsequences with respect to their profiles by grouping the elements of a plurality of subsequences having a measure of similarity.

10. The method of claim 9 wherein the clustering is according to a k-clustering algorithm.

11. The method of claim 9 wherein the clustering is according to the following algorithm:
Given N vectors {Aᵢ}ᵢ^{N}=1, a threshold λ and a perturbation vector e(B), J clusters (where the target number of the clusters, J, is a power of 2) are obtained as follows:
1. set
$${B}_{1} = \frac{1}{N} {\sum }_{i = 1}^{N} {A}_{i} ,$$
2. for each Bₖ, k=1, . . . ,N, if k=J, stop; otherwise, let Bₖ₊₁=e(Bₖ),i=1, . . . ,k. D₀=0,
3. determine Cⱼ={Aₙ:d(Aₙ,Bⱼ)<d(Aₙ,Bᵢ)∀i≠j},j=1, . . . ,2k.
4. determine
$${D}_{n} = {\sum }_{j = 1}^{2 k} {\sum }_{i = 1}^{\vert {c}_{j} \vert} ⅆ ( {B}_{j} , {A}_{i j} ) ,$$
where Aᵢⱼ is the i^{th} vector in the j^{th} cluster, Cⱼ;
5. if
$$\frac{{D}_{n} - {D}_{0}}{{D}_{n}} < λ ,$$
go to step 2, otherwise continue;
6. D₀=Dₙ, find new representation vectors Bⱼ that are the average value of the vectors in the j^{th} cluster Cⱼ; go to step 3;
where d(A,B) is the Euclidean distance between the vectors A and B.

12. The method of claim 9 wherein the clustering is according to the following algorithm:
Given N vectors
$${\{ {A}_{i} \}}_{i}^{N} = 1 ,$$
a threshold λ and a perturbation vector e(B), J clusters (where the target number of the clusters, J, is not a power of 2) are obtained as follows:
1. set
$${B}_{1} = \frac{1}{N} {\sum }_{i = 1}^{N} {A}_{i} ,$$
2. For each Bₖ, k=1, . . . ,N, if k=J, stop; otherwise, let Bₖ₊₁=e(Bₖ),i=1, . . . ,k. D₀=0,
3. determine Cⱼ={Aₙ:d(Aₙ,Bⱼ)<d(Aₙ,Bᵢ)∀i≠j}, j=1, . . . ,2k.
4. determine
$${D}_{n} = {\sum }_{j = 1}^{2 k} {\sum }_{i = 1}^{\vert {c}_{j} \vert} ⅆ ( {B}_{j} , {A}_{i j} ) ,$$
where Aᵢⱼ is the i^{th} vector in the j^{th} cluster, Cⱼ;
5. if
$$\frac{{D}_{n} - {D}_{0}}{{D}_{n}} < λ ,$$
go to step 2, otherwise continue;
6. for D₀=Dₙ, find new representation vectors Bⱼ that are the average value of the vectors in the j^{th} cluster Cⱼ; go to step 3;
where d(A,B) is the Euclidean distance between the vectors A and B.

13. The method of claim 10 wherein the k-clustering algorithm is a k-means algorithm which partitions the fragments into J clusters.

14. The method of claim 13 further comprising the steps of identifying subgroups of each of the J clusters which do not overlap and eliminating the identified subgroups.

15. The method of claim 9 wherein the step of determining a profile for each sequence comprises determining a profile of an Average Mutual Information (AMI) for each subsequence wherein the Average Mutual Information is defined by the following function:
$$I ( r ) = {\sum }_{i . j} {p}_{i j} ( r ) {log}_{2} ( \frac{{p}_{i j} ( r )}{{p}_{i} {p}_{j}} )$$
where pᵢ is the density of the symbol i and pᵢⱼ(r) is the joint probability of observing symbol i and j separated by a distance r and where I(r) is the amount of information symbol i carries about symbol j at a distance r.

16. The method of claim 1 wherein the consensus sequences are considered second generation clusters comprising a new collection of subsequences and wherein the steps of assigning, clustering, and aligning are recursively applied to the new collection of subsequences.

17. The method of claim 1 wherein subgroups are generated as follows:
1. put fₖ in Cⱼ^{k}, k=1, . . . ,|Cⱼ|, set i=1,
2. if s(fᵢ,fⱼ)>λ(fᵢ,fⱼ), or s(fᵢ\overline{,}fⱼ)>λ(fᵢ\overline{,}fⱼ) combine the subgroup that contains fⱼ with the subgroup that contains fᵢ, do this for j=i+1, . . . , |Cⱼ|,
3. increment i; if i=|Cⱼ|, stop; otherwise, go to step 2,
where s(fᵢ,fⱼ) is the score of the optimal semiglobal alignment

18. The method of claim 1 further comprising a multiple alignment technique for aligning the consensus sequences in a cluster in which there is an interlacing of the sequences in each cluster according to the following algorithm:
1. Find the pairwise alignments of the sequences in the cluster;
2. Represent each alignment as an interlacing of the two sequences involved;
3. Create a graph where nodes represent sequences and the weight of an edge connecting two nodes represents the score of the alignment between the sequences;
4. Find a maximum spanning tree of this graph; and
5. Combine the pairwise alignment implied by each edge with the pairwise alignment implied by the following edge while moving through the graph.

19. A system of assembling subsequences of a DNA sequence comprising:
a database of the subsequences to be assembled; and
a processor accessing the subsequences in the database and having software for:
assigning a numerical characterization to each subsequence, each numerical characterization comprising a set of numbers;
clustering the sets; and
aligning the sets to form a consensus sequence for each cluster.

20. A system of assembling subsequences of a DNA sequence comprising:
a database of the subsequences to be assembled; and
a processor accessing the subsequences in the database and having software for:
assigning a numerical characterization to each subsequence, each numerical characterization comprising a set of numbers;
defining a vector for each subsequence, each vector having coordinates corresponding to the numerical characterizations of one subsequence;
clustering the vectors; and
aligning the vectors, wherein said aligning comprises comparing the vectors in each cluster and aligning the subsequences of corresponding compared vectors to form a consensus sequence for each cluster.
